(19) United States
(12) Patent Application Publication
(10) Pub. No.: US 2025/0179092 A1
(43) Pub. Date: Jun. 5, 2025

(54) PYRIDO[1,2-A]PYRIMIDIN-4-ONE DERIVATIVES

(71) Applicant: Hoffmann-La Roche Inc., Little Falls, NJ (US)

(72) Inventors: Virginie Brom, Blotzheim (FR); Cosimo Dolente, Allschwil (CH); Delphine Gaufreteau, Kembs (FR); Fionn Susannah O'Hara, Basel (CH); Matilde Piras, Basel (CH); Hasane Ratni, Habsheim (FR); Michael Reutlinger, Freiburg (DE); Walter Vifian, Sissach (CH); Claudio Zambaldo, Basel (CH)

(73) Assignee: Hoffmann-La Roche Inc., Little Falls, NJ (US)

(21) Appl. No.: 18/844,371

(22) PCT Filed: Mar. 8, 2023
(86) PCT No.: PCT/EP2023/055838

§ 371 Date: Sep. 5, 2024

(30) Foreign Application Priority Data
Mar. 10, 2022 (EP) 22161258.3

(51) Int. Cl. C07D 519/00; A61K 31/519

(52) U.S. Cl. CPC C07D 519/00; A61K 31/519

(57) ABSTRACT

The invention relates to a compound of formula (I) wherein R¹-R³ and A₁-A₂ are as defined in the description and in the claims. The compound of formula (I) can be used as a medicament.

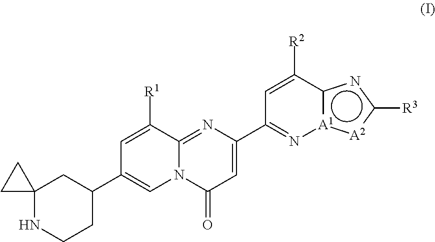

The present invention relates to new organic compounds useful for therapy and/or prophylaxis in a mammal, and in particular to compounds that reduce the protein level of huntingtin (HTT) and which are useful in the treatment of Huntington's disease.

In particular, the present invention relates to a compound of formula (I)

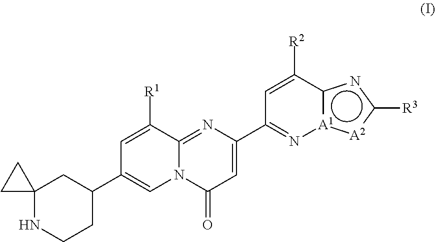

wherein
R¹ is hydrogen or alkyl;
R² is hydrogen, halogen, alkyl, alkoxy or haloalkyl;
R³ is hydrogen, alkyl or halogen;
A¹ is —N— or —C—; and
A² is —CH— or —O—; with the proviso that if A¹ is —N—, then A² is —CH—; and the proviso that if A¹ is —C—, then A² is —O—;
or a pharmaceutically acceptable salt thereof.

Huntington's Disease (HD) is an inherited autosomal dominant neurodegenerative disease caused due to a CAG bases repeat expansion in the huntingtin (HTT) gene. Several lines of evidence indicate that the mutant HTT gene together with its gene product mHTT protein contributes to HD pathogenesis via a toxic gain of function mechanism.

The triplet repeat expansion in the exon 1 of the HTT gene translates into a polyglutamine repeat in the HTT protein which is prone to misfolding and aggregating in the cells. While the exact mechanisms of how mutant HTT disrupts cellular function is unclear, several processes ranging from interruption of RNA translation, toxic RNA species, protein aggregates, RNA translation, and stress granules have been implicated.

At a neural circuit level, HD has been shown to affects deep brains structures like the striatum as well as cortical regions to different extents. Seminal mouse genetic experiments coupled with human imaging experiments point to a key role of cortico-striatal connections in the pathogenicity of HD (Wang et al., “Neuronal targets of mutant huntingtin genetic reduction to ameliorate Huntington's disease pathogenesis in mice” Nature medicine 20.5 (2014): 536; Tabrizi et al.; “Potential endpoints for clinical trials in premanifest and early Huntington's disease in the TRACK-HD study: analysis of 24 month observational data.” The Lancet Neurology 11.1 (2012): 42-53).

HD typically manifests around 30-50 years of age characterized by a multitude of symptoms spanning the motor, cognitive and affective domains eventually leading to death in 10-20 years after the onset of motor symptoms. CAG repeat length negatively correlates with age of onset of motor symptoms, however this only accounts for 50-70% of the variance in age of onset. In an effort to identify genetic modifiers of age of onset in HD, Lee et al. (2019, Huntington's disease onset is determined by length of uninterrupted CAG, not encoded polyglutamine, and is modified by DNA maintenance mechanisms. Bioarxiv doi: https://doi.org/10.1101/529768) conducted a large GWAS (genome-wide association study) that has uncovered additional genetic modifiers of age of onset.

Various mouse models have been characterized to model aspects of HD. The YAC128 mice expressing the full length mutant HTT transgene with 128 CAG repeats, BACHD mice expressing the full length mutant HTT genomic sequence with 97 CAG/CAA repeats, the R6/2 mice expressing exon 1 of the mutant human HTT gene with 110-135 CAG repeats). In addition to these mice that express the human transgene, there are also a series of mouse models, like the frequently used Q111, the Q175 knock in mice where the expanded repeats are knocked-in in the context of the mouse HTT locus.

There are currently no disease modifying therapies for Huntington's Disease while several are in development. The core disease process behind the symptomatology characterized by motor, cognitive and behavioral symptoms remains unmet by the various symptomatic treatments currently approved. Tetrabenazine and tiapride are currently approved for the treatment of motor symptoms namely HD-associated chorea. In addition, anticonvulsants, benzodiazepines, antidepressants, and antipsychotics are also used off-label to treat the motor, cognitive and psychiatric symptoms associated with HD.

Several therapeutic strategies targeting DNA and RNA are being investigated for HTT lowering (E. J. Wild, S. Tabrizi, Lancet Neurol. 2017 16(10): 837-847). HTT lowering is a promising therapeutic approach that aims to slow disease progression by getting at the core cause of Huntington's Disease. HTT lowering is thought to be transformative when treated in the premanifest or manifest stages of disease onset, thus preventing major neurodegenerative processes in the brain. However, the challenge lies in identifying the patients at the right disease stage, as age of onset is quite variable across the population (S. J. Tabrizi, R. Ghosh, B. R. Leavitt, Neuron, 2019, 102(4), 899).

The current clinical approaches are mainly based on antisense oligonucleotides (ASOs). In addition, a few allele specific lowering strategies such as SNP (single-nucleotide polymorphism) based ASO and zinc finger based gene editing approaches are investigated. While the use of small molecules to lower HTT expression has been postulated, this strategy has not yet been validated and none has proved successful so far.

Small molecules provide an opportunity to allow for HTT lowering in the brain as well as the periphery. In addition, a small molecule modality allows access to patient populations that could be difficult to reach with modalities like ASOs or gene therapy.

There is thus the need for new compounds capable of lowering mHTT.

The applicant has surprisingly found that the compounds of the invention are active in lowering mHTT and are therefore useful in the treatment of HD.

All publications, patent applications, patents, and other references mentioned herein are incorporated by reference in their entirety.

In the present description the term “alkyl”, alone or in combination, signifies a linear or branched saturated hydrocarbon group of 1 to 8 carbon atoms, in particular of 1 to 6 carbon atoms and more particular of 1 to 4 carbon atoms. Examples of straight-chain and branched-chain C1-C8 alkyl groups are for instance methyl, ethyl, propyl, isopropyl, butyl, isobutyl, tert.-butyl, the isomeric pentyls, the isomeric hexyls, the isomeric heptyls and the isomeric octyls. Particular examples of “alkyl” are methyl, ethyl and isopropyl. Methyl and ethyl are particular examples of “alkyl” in the compound of formula (I).

The term “alkoxy” or “alkyloxy”, alone or in combination, signifies a group of the formula alkyl-O— in which the term “alkyl” has the previously given significance. Examples of alkoxy are for instance methoxy, ethoxy, n-propoxy, isopropoxy, n-butoxy, isobutoxy, sec-butoxy and tert.-butoxy. A particular example of “alkoxy” is methoxy.

The term “oxy”, alone or in combination, signifies the —O— group.

The terms “halogen” or “halo”, alone or in combination, signifies fluorine, chlorine, bromine or iodine and particularly fluorine, chlorine or bromine. One preferred example of halogen is fluorine. The term “halo”, in combination with another group, if not otherwise specified, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Particular “haloalkyl” are fluoromethyl, trifluoromethyl, difluoromethyl, fluoroethyl, fluoropropyl and fluorobutyl. Further particular “haloalkyl” are difluoromethyl and trifluoromethyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and trifluoroacetic acid. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts formed with trifluoroacetic acid or hydrochloric acid.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3rd Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), trityl (Trt), 2,4.dimethoxybenzyl (Dmb), 9-fluorenylmethyl carbamate (Fmoc), 2-trimethylsilylethyl carbamate (Teoc), carbobenzyloxy (Cbz) and p-methoxybenzyloxycarbonyl (Moz). A particular example of a protecting group is tert-butoxycarbonyl (Boc).

A certain embodiment of the invention relates to the compound of formula (I) as described herein, or a pharmaceutically acceptable salt thereof, wherein at least one substituent comprises at least one radioisotope. Particular examples of radioisotopes are ²H, ³H, ¹³C, ¹⁴C and ¹⁸F.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

The compound of formula (I) may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

The term “asymmetric carbon atom” means a carbon atom with four different substituents. According to the Cahn-Ingold-Prelog Convention an asymmetric carbon atom can be of the “R” or “S” configuration.

The invention thus also relates in particular to:

A compound according to the invention, wherein R¹ is hydrogen or methyl;

A compound according to the invention, wherein R¹ is hydrogen;

A compound according to the invention, wherein R¹ is methyl;

A compound according to the invention, wherein R² is hydrogen, alkyl, alkoxy or haloalkyl;

A compound according to the invention, wherein R² is hydrogen, methyl, methoxy or trifluoromethyl;

A compound according to the invention, wherein R² is hydrogen;

A compound according to the invention, wherein R² is methyl;

A compound according to the invention, wherein R² is methoxy;

A compound according to the invention, wherein R² is trifluoromethyl;

A compound according to the invention, wherein R³ is alkyl or halogen;

A compound according to the invention, wherein R³ is methyl or chloro;

A compound according to the invention, wherein R³ is methyl;

A compound according to the invention, wherein R³ is chloro;

A compound according to the invention, wherein A₁ is —N—, and A² is —CH—;

A compound according to the invention, wherein A₁ is —C—, and A₂ is —O—; and

A compound of formula (I) according the invention selected from
rac-7-(4-azaspiro[2.5]octan-7-yl)-2-(2,8-dimethylimidazo[1,2-b]pyridazin-6-yl)pyrido[1,2-a]pyrimidin-4-one;
rac-7-(4-azaspiro[2.5]octan-7-yl)-2-(8-methoxy-2-methyl-imidazo[1,2-b]pyridazin-6-yl)pyrido[1,2-a]pyrimidin-4-one;
rac-7-(4-azaspiro[2.5]octan-7-yl)-2-[2-methyl-8-(trifluoromethyl)imidazo[1,2-b]pyridazin-6-yl]pyrido[1,2-a]pyrimidin-4-one;
rac-7-(4-azaspiro[2.5]octan-7-yl)-2-(2,7-dimethyloxazolo[5,4-b]pyridin-5-yl)pyrido[1,2-a]pyrimidin-4-one; and
rac-7-(4-azaspiro[2.5]octan-7-yl)-2-(2-chloroimidazo[1,2-b]pyridazin-6-yl)pyrido[1,2-a]pyrimidin-4-one;
or a racemic mixture or its corresponding enantiomers thereof,
or a pharmaceutically acceptable salt thereof.

In one embodiment of the invention the compound of formula (I) is a compound of formula (Ia)

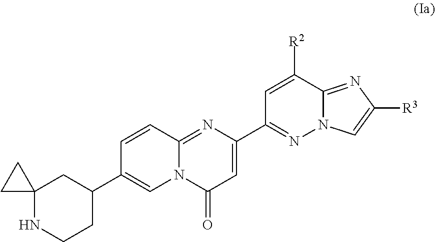

wherein
R² is hydrogen, alkyl, alkoxy or haloalkyl;
R³ is hydrogen, alkyl or halogen;
or a pharmaceutically acceptable salt thereof.

In one embodiment of the invention the compound of formula (I) is a compound of formula (Ib)

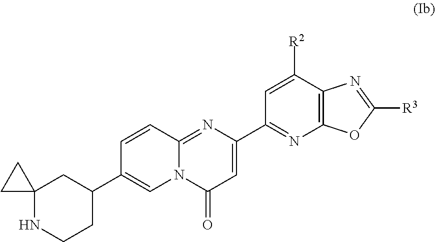

wherein
R² is hydrogen, alkyl, alkoxy or haloalkyl, in particular alkyl;
R³ is hydrogen, alkyl or halogen, in particular alkyl;
or a pharmaceutically acceptable salt thereof.

The synthesis of the compound of formula (I) can, for example, be accomplished according to the following schemes. R¹-R³ and A₁-A₂ are as defined above, unless otherwise specified.

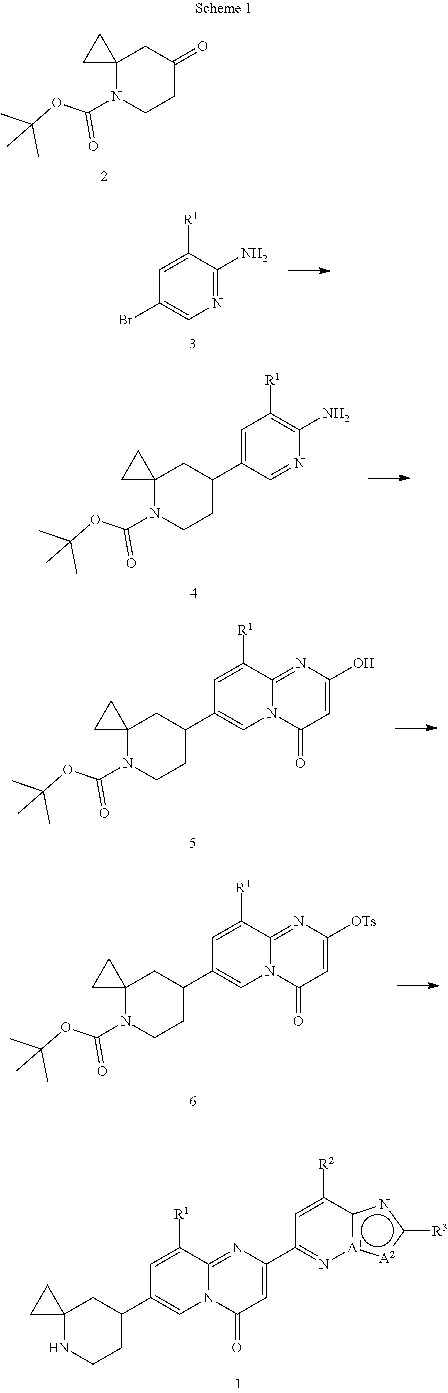

The preparation of derivatives of general formula 1, can be made according to the general scheme 1. A Suzuki coupling was performed between tert-butyl 7-oxo-4-azaspiro[2.5]octane-4-carboxylate 2 and an amino pyridine compound 3 in the presence of 4-methylbenzenesulfonohydrazide, palladium and a ligand. The resulting intermediate was then readily hydrogenated with Pd/C and H₂ to afford derivatives 4. Upon condensation with malonic acid bis(2,4,6-trichlorophenyl) ester, the 2-hydroxypyrido[1,2-a]pyrimidin-4-one derivatives 5 were obtained. Those compounds 5 reacted with p-toluenesulfonyl chloride to form 6. Finally a Suzuki coupling between derivatives 6 and a suitable boronic acid/ester yielded after Boc-deprotection the final compounds of general formula 1.

The invention thus also relates to a process for the preparation of a compound according to the invention, comprising at least one of the following steps:
(a) the reaction of a compound of formula (B1)

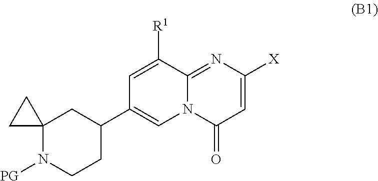

with a compound of formula (B2)

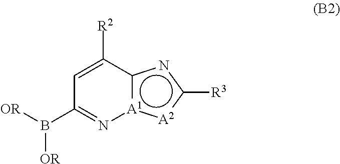

in a suitable solvent in the presence of a base and a suitable palladium catalyst, wherein n is 0 or 1, X is O-tosylate, O-triflate, O-mesylate or halogen, and wherein in —B(OR)₂ each R is independently selected from hydrogen and alkyl, or —B(OR)₂ is optionally substituted dioxaborolanyl, to arrive at a compound of formula (B3)

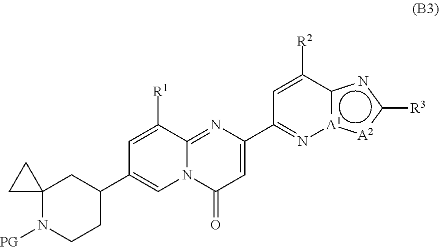

(b) the reaction of the compound of formula (B3), in a suitable solvent and in presence of an acid to yield the compound of formula (I)

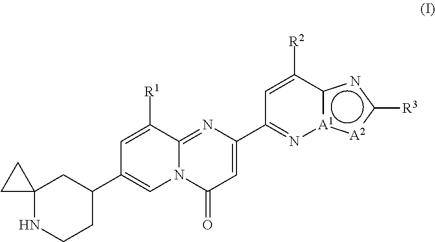

wherein in the process R¹, R², R³, A₁ and A₂ are as defined above, and PG is a protecting group.

The reaction of step (a) can be conveniently carried out in a solvent. The solvent can be for example 1,4-dioxane, acetonitrile, water or a mixture thereof,

In the reaction of step (a), the base can be for example K₂CO₃, Li₂CO₃, Na₂CO₃, KOtBu, Cs₂CO₃, NaOtBu or LiOtBu, in particular K₂CO₃;

In the reaction of step (a), the palladium catalyst can be for example Pd(dppf)Cl₂·CH₂Cl₂ (0.2 eq. CAS #95464-05-4) or XPhos PdG4 CAS #1599466-81-5;

In the reaction of step (a), X is conveniently 0-tosylate or chloro, in particular O-tosylate;

In the reaction of step (a), B(OR)₂ can be for example dioxaborolanyl optionally substituted with one, two, three or four alkyl, in particular 4,4,5,5-tetramethyl-1,3,2-dioxaborolan-2-yl;

Convenient conditions for the reaction of step (a) are around 20° C.-150° C., particularly around 40° C.-130° C., more particularly around 60° C.-110° C., in particular around 90° C.; Particular conditions for the reaction of step (a) are the use of K₂CO₃ in 1,4-dioxane, acetonitrile, water or a mixture thereof at around 90° C. for around 2 hrs-8 hrs;

The reaction of step (b) can be conveniently carried out in a solvent. The solvent can be for example CH₂Cl₂ or 1,4-dioxane;

In the reaction of step (b) the acid can be for example TFA or HCl;

Convenient conditions for the reaction of step (b) are around 0° C.-100° C., particularly around 5° C.-80° C., more particularly around 10° C.-60° C., in particular around 15° C.-40° C.; Particular conditions for the reaction of step (b) are the use of TFA in CH₂Cl₂ at around 15-40° C. for around 1 hrs-24 hrs, in particular for around 1 h-3 hrs;

In the process, the protecting group can be for example Boc, Trt or Dmb, in particular Boc.

The invention also relates to a compound according to the invention when manufactured according to a process of the invention.

The invention thus also relates in particular to:

A compound according to the invention for use as therapeutically active substance;

A pharmaceutical composition comprising a compound according to the invention and a therapeutically inert carrier;

A compound according to the invention for use in the treatment or prophylaxis of a neurodegenerative disease;

A compound according to the invention for use in the treatment or prophylaxis of Huntington's disease;

The use of a compound according to the invention for the treatment or prophylaxis of a neurodegenerative disease, in particular Huntington's disease;

The use of a compound according to the invention for the preparation of a medicament for the treatment or prophylaxis of a neurodegenerative disease, in particular Huntington's disease; and

A method for the treatment or prophylaxis of a neurodegenerative disease, in particular Huntington's disease, which method comprises administering an effective amount of a compound according to the invention to a patient in need thereof.

A certain embodiment of the invention relates to a pharmaceutical composition comprising the compound of formula (I) as described herein, or a pharmaceutically acceptable salt thereof, and a pharmaceutically acceptable auxiliary substance.

Furthermore, structures depicted herein are also meant to include compounds that differ only in the presence of one or more isotopically enriched atoms. For example wherein one or more hydrogen atoms are replaced by deuterium (2H), or one or more carbon atoms are replaced by a 13C- or 14C-enriched carbon are within the scope of this invention.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

The compound of formula (I) may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

Also an embodiment of the present invention is a compound of formula (I) as described herein, when manufactured according to any one of the described processes.

The compound of formula (I) or a pharmaceutically acceptable salt thereof can be used as a medicament (e.g. in the form of a pharmaceutical preparation). The pharmaceutical preparation can be administered internally, such as orally (e.g. in the form of tablets, coated tablets, dragées, hard and soft gelatin capsules, solutions, emulsions or suspensions), nasally (e.g. in the form of nasal sprays), rectally (e.g. in the form of suppositories) or topical ocularly (e.g. in the form of solutions, ointments, gels or water soluble polymeric inserts). However, the administration can also be effected parenterally, such as intramuscularly, intravenously, or intraocularly (e.g. in the form of sterile injection solutions).

The compound of formula (I) or a pharmaceutically acceptable salt thereof can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparation can contain preservatives, solubilizers, viscosity-increasing substances, stabilizers, wetting agents, emulsifiers, sweeteners, colorants, flavorants, salts for varying the osmotic pressure, buffers, masking agents or antioxidants. The pharmaceutical preparation can also contain still other therapeutically valuable substances.

The dosage can vary in wide limits and will be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

### Pharmaceutical Compositions

The compound of formula (I) or a pharmaceutically acceptable salt thereof can be used as a therapeutically active substance, e.g. in the form of a pharmaceutical preparation. The pharmaceutical preparation can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compound of formula (I) or a pharmaceutically acceptable salt thereof can be processed with pharmaceutically inert, inorganic or organic carriers for the production of a pharmaceutical preparation. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparation can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula (I) or a pharmaceutically acceptable salt thereof and a therapeutically inert carrier are also provided by the present invention, as is a process for their production, which comprises bringing a compound of formula (I) and/or pharmaceutically acceptable salts thereof and, if desired, one or more other therapeutically valuable substances into a galenical administration form together with one or more therapeutically inert carriers.

The dosage can vary within wide limits and will, have to be adjusted to the individual requirements in each particular case. In the case of oral administration the dosage for adults can vary from about 0.01 mg to about 1000 mg per day of a compound of general formula (I) or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparation conveniently contains about 1-500 mg, particularly 1-100 mg, of a compound of formula (I). Examples of compositions according to the invention are:

### EXAMPLE A

Tablets of the following composition are manufactured in the usual manner:

TABLE 1
possible tablet composition

mg/tablet

| ingredient | 5 | 25 | 100 | 500 |
|---|---|---|---|---|
| Compound of formula (I) | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### EXAMPLE B-1

Capsules of the following composition are manufactured:

TABLE 2
possible capsule ingredient composition

mg/capsule

| ingredient | 5 | 25 | 100 | 500 |
|---|---|---|---|---|
| Compound of formula (I) | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### EXAMPLE B-2

Soft Gelatin Capsules of the following composition are manufactured:

TABLE 3
possible soft gelatin capsule ingredient composition

| ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 4
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### EXAMPLE C

Suppositories of the following composition are manufactured:

TABLE 5
possible suppository composition

| ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### EXAMPLE D

Injection solutions of the following composition are manufactured:

TABLE 6
possible injection solution composition

| ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### Manufacturing Procedure

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### EXAMPLE E

Sachets of the following composition are manufactured:

TABLE 7
possible sachet composition

| ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### EXAMPLES

### Abbreviations

AcOEt: ethyl acetate; AcOH: acetic acid; DCM: dichloromethane; DMAP: 4-dimethylaminopyridine; DMSO: dimethyl sulfoxide; ES+: positive electrospray ionization; EtOAc: ethyl acetate; EtOH: ethanol; HPLC: high performance liquid chromatography; HTRF: homogeneous time resolved fluorescence; MeOH: methanol; MS: mass spectrometry; PPTS: pyridinium p-toluenesulfonate; RT: room temperature; TFA: trifluoroacetic acid.

The following examples are provided for illustration of the invention. They should not be considered as limiting the scope of the invention, but merely as being representative thereof.

### Intermediates

### Preparation of Boronic Ester or Acid

Those derivatives were ultimately obtained as either a boronic ester, boronic acid or a mixture thereof and used directly in the subsequent step.

### Boronic Ester 1

### 2,8-dimethyl-6-(4,4,5,5-tetramethyl-1,3,2-dioxaborolan-2-yl)imidazo[1,2-b]pyridazine

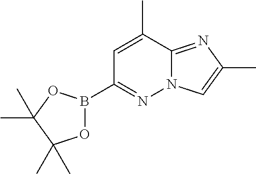

Prepared according to WO2019/057740

### Boronic Ester 2

### 8-methoxy-2-methyl-6-(4,4,5,5-tetramethyl-1,3,2-dioxaborolan-2-yl)imidazo[1,2-b]pyridazine

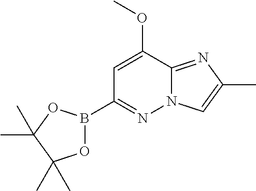

### Step 1: Preparation of 8-bromo-6-chloro-2-methyl-imidazo[1,2-b]pyridazine

To a solution of (4-bromo-6-chloro-pyridazin-3-yl)amine (2000 mg, 9.6 mmol) and PPTS (241 mg, 0.96 mmol) in isopropanol (19 mL) was added 1-bromo-2,2-dimethoxy-propane (2.11 g, 1.56 mL, 11.5 mmol) at RT. The reaction mixture was heated at reflux for 30 hours. After cooling down to RT, the mixture was partitioned between ethyl acetate (50 ml) and 1M Na₂CO₃ sol (30 ml). The layers were separated, and the organic layer was washed with one 30-ml portion of brine and dried over sodium sulfate, filtered and concentrated in vacuo to give the crude title compound (2.37 g, 92% yield) as light brown solid with a purity of 92%, which was used without further purification. MS (ES+) m/z: 246.0-248.0 [(M+H)⁺].

### Step 2: Preparation of 6-chloro-8-methoxy-2-methyl-imidazo[1,2-b]pyridazine

To a solution of 8-bromo-6-chloro-2-methyl-imidazo[1,2-b]pyridazine (500 mg, 2.03 mmol) and cesium carbonate (1.4 g, 4.3 mmol) in acetonitrile (10 mL) was added MeOH (400 uL, 9.89 mmol) at RT and stirring was continued for 4 hours. The mixture was partitioned between ethyl acetate (30 mL) and water (30 mL). The combined organic layer was washed with one 30-mL portion of brine, dried over sodium sulfate, filtered and concentrated in vacuo. A purification by flash gave 6-chloro-8-methoxy-2-methyl-imidazo[1,2-b]pyridazine (337 mg; 84% yield) as a white solid. MS (ES+) m/z: 198.0 [(M+H)⁺].

### Step 3: Preparation of 8-methoxy-2-methyl-6-(4,4,5,5-tetramethyl-1,3,2-dioxaborolan-2-yl)imidazo[1,2-b]pyridazine

To a mixture of 6-chloro-8-methoxy-2-methyl-imidazo[1,2-b]pyridazine (118 mg, 0.597 mmol), bis(pinacolato)diboron (151.5 mg, 0.597 mmol, 1 eq) and potassium acetate (150.48 mg, 1.53 mmol) in 1,4-dioxane (1.2 mL) was added of XPhos Pd G4 (22 mg, 0.026 mmol). The reaction mixture was heated at 100° C. for 1 hour. The reaction mixture was cooled to RT and diluted with ethyl acetate (5-10 mL). The solids were removed by filtration. The filtrate was concentrated in vacuo to give the crude title compound which was used directly in the next step without further purification.

### Boronic Ester 3

### 2-methyl-6-(4,4,5,5-tetramethyl-1,3,2-dioxaborolan-2-yl)-8-(trifluoromethyl)imidazo[1,2-b]pyridazine

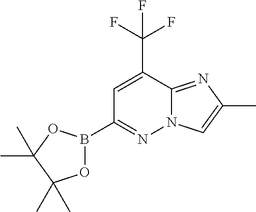

### Step 1: Preparation of 6-chloro-2-methyl-8-(trifluoromethyl)imidazo[1,2-b]pyridazine

In a 10 mL round bottom flask, equipped with a magnetic stirrer bar, reflux condenser and N2-inlet bubbler, [6-chloro-4-(trifluoromethyl)pyridazin-3-yl]amine (94 mg, 0.476 mmol) and pyridinium p-toluene sulfonate (11.9 mg, 0.048 mmol) were combined with isopropanol (2 mL). 1-bromo-2,2-dimethoxy-propane (104.51 mg, 77.13 uL, 0.571 mmol, 1.2 eq) was added and the colorless solution was stirred 24 hours at 75° C. The resulting dark-brown reaction mixture was cooled to RT and diluted with EtOAc (10 mL) and washed with a saturated aqueous NaHCO₃-solution (10 mL). Organic layer was separated and dried over sodium sulfate, filtered off and concentrated in vacuo. The crude (120 mg brownish viscous oil) was purified by column chromatography yielding 6-chloro-2-methyl-8-(trifluoromethyl)imidazo[1,2-b]pyridazine (46 mg, 34% yield) as light yellow solid. MS (ES+) m/z: 236.1 [(M+H)⁺].

### Step 2: Preparation of 2-methyl-6-(4,4,5,5-tetramethyl-1,3,2-dioxaborolan-2-yl)-8-(trifluoromethyl)imidazo[1,2-b]pyridazine

In a dry/Argon flushed 20 mL microwave tube with a magnetic stirrer bar and a cap-septum, 6-chloro-2-methyl-8-(trifluoromethyl)imidazo[1,2-b]pyridazine (300 mg, 1.2 mmol), bis(pinacolato)diboron (364.7 mg, 1.44 mmol) and potassium acetate (352.42 mg, 3.59 mmol) were combined with 1,4-dioxane (12 mL). The yellowish fine suspension was stirred and degassed with Argon for 10-15 minutes before tetrakis(triphenylphosphine)palladium (69.1 mg, 0.060 mmol) was added. The vial was sealed and stirred in a heating block (Temperature: 100° C.) for 22 hours. Further addition of tetrakis(triphenylphosphine)palladium (69 mg, 0.060 mmol), after 90 minutes, 3.5 hours and 6 hours. The reaction was cooled to room temperature, filtered off and concentrated in vacuo. The amber viscous oil was purified by column chromatography to give 2-methyl-6-(4,4,5,5-tetramethyl-1,3,2-dioxaborolan-2-yl)-8-(trifluoromethyl)imidazo[1,2-b]pyridazine (428 mg, 48%) as yellow viscous oil.

### Boronic Ester 4

### 2,7-dimethyl-5-(4,4,5,5-tetramethyl-1,3,2-dioxaborolan-2-yl)oxazolo[5,4-b]pyridine

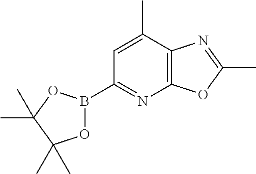

### Step 1: Preparation of N-(2,6-dichloro-4-methyl-3-pyridyl)acetamide

To a solution of (2,6-dichloro-4-methyl-3-pyridyl)amine (2000 mg, 11.3 mmol) and Ac₂O (11.53 g, 10.66 mL, 112.98 mmol) was added a catalytic amount of DMAP. The reaction mixture was stirred for 1 hour at RT then over night at 60° C. The reaction mixture was cooled to RT, and ethanol (10 mL, 169.46 mmol) was slowly added and stirring continued for 30 min. The solvent were evaporated and residual AcOH was removed by azeotropic distillation with one 30-mL portion of toluene. The residue was triturated in warm ethyl acetate (15 mL). Heptane (15 mL) was added and the suspension was stirred for 1 h. The precipitate was collected by filtration, washed with two 10 mL-portions of ethyl acetate/n-heptane (1:1) and dried in vacuo to give N-(2,6-dichloro-4-methyl-3-pyridyl)acetamide (1750 mg, 71% yield) as brown solid. MS (ES+) m/z: 219.1 [(M+H)⁺].

### Step 2: Preparation of 5-chloro-2,7-dimethyl-oxazolo[5,4-b]pyridine

To a solution of N-(2,6-dichloro-4-methyl-3-pyridyl)acetamide (1750 mg, 7.99 mmol) in N-methyl-2-pyrrolidinone (16 mL) was added NaH, 55% in oil (348.58 mg, 7.99 mmol) at RT. The reaction mixture was heated at 120° C. and stirred for 20 hours. After cooled down to RT, acetic acid (959 mg, 914 uL, 15.98 mmol) was slowly added and stirred for an additional 30 min. The mixture was partitioned between ethyl acetate (50 mL) and 1M NaHCO₃ sol (20 mL). The organic layer was washed with one 50-mL portion of water and one 30-mL portion of brine, dried over sodium sulfate, filtered and concentrated in vacuo. A purification by flash chromatography gave 5-chloro-2,7-dimethyl-oxazolo[5,4-b]pyridine (85 mg, 6% yield) as a white solid. MS (ES+) m/z: 183.1 [(M+H)⁺].

### Step 3: 2,7-dimethyl-5-(4,4,5,5-tetramethyl-1,3,2-dioxaborolan-2-yl)oxazolo[5,4-b]pyridine

The title compound was prepared from 5-chloro-2,7-dimethyl-oxazolo[5,4-b]pyridine in analogy to the synthesis of the boronic ester 5, step 2.

### Boronic Ester 5

### 2-chloro-6-(4,4,5,5-tetramethyl-1,3,2-dioxaborolan-2-yl)imidazo[1,2-b]pyridazine

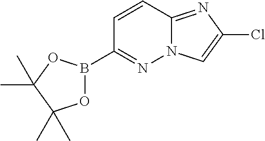 [SMILES: CC1(C)OB(c2ccc3nc(Cl)cn3n2)OC1(C)C]

In analogy to the boronic ester 1, from 2,6-dichloroimidazo[1,2-b]pyridazine, the title compound was prepared.

### Example 1

### rac-7-(4-azaspiro[2.5]octan-7-yl)-2-(2,8-dimethylimidazo[1,2-b]pyridazin-6-yl)pyrido[1,2-a]pyrimidin-4-one

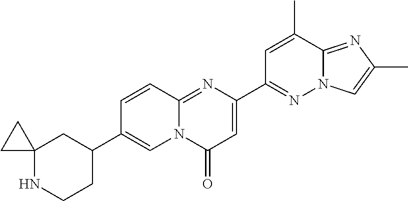 [SMILES: Cc1cn2nc(-c3cc(=O)n4cc(C5CCNC6(CC6)C5)ccc4n3)cc(C)c2n1]

Step 1: In a three necked flask purged with argon, 7-keto-4-azaspiro[2.5]octane-4-carboxylic acid tert-butyl ester (781 mg, 3.47 mmol, 1.2 eq) and 4-methylbenzenesulfonohydrazide (700 mg, 3.76 mmol, 1.3 eq) were dissolved in degassed 1,4-dioxane, extra dry (10 mL). Then 2-amino-5-bromopyridine (500 mg, 2.89 mmol, 1 eq), Lithium Tert-butoxide (810 mg, 10.12 mmol, 3.5 eq), X-PHOS (138 mg, 0.289 mmol, 0.100 eq) and Bis(Dibenzylideneacetone) Palladium (83 mg, 0.145 mmol, 0.050 eq) were added at room temperature. The mixture was degassed 3 times with argon and stirred for 16 hours at 110° C. The reaction mixture was diluted with saturated NaHCO₃-solution and extracted two times with EtOAc. The organic layers were washed with water and brine, dried over Na₂SO₄ and concentrated to dryness. The crude material was purified by flash chromatography on silica gel (40 g, AcOEt in Heptan 0 to 100%) to afford tert-butyl 7-(6-amino-3-pyridyl)-4-azaspiro[2.5]oct-7-ene-4-carboxylate and/or tert-butyl 7-(6-amino-3-pyridyl)-4-azaspiro[2.5]oct-6-ene-4-carboxylate (550 mg, 62% yield) as light brown solid. MS (ES+) m/z: 302.2 [(M+H)⁺].

Step 2: The product from step 1 (550 mg, 1.82 mmol, 1 eq) was dissolved in methanol (8 mL) and palladium on carbon (194 mg, 0.182 mmol, 0.1 eq) was added at room temperature. The mixture was stirred for 24 hours under H₂ vigorously. The reaction mixture was then flushed with N₂, filtered through dicalite, washed with MeOH and the solvent was concentrated to dryness. The crude material was purified by flash chromatography on silica gel (40 g, MeOH in DCM 0 to 10%) to afford rac-tert-butyl 7-(6-amino-3-pyridyl)-4-azaspiro[2.5]octane-4-carboxylate (470 mg, 85.43%) as colorless oil. MS (ES+) m/z: 304.2 [(M+H)⁺].

Step 3: rac-tert-butyl 7-(6-amino-3-pyridyl)-4-azaspiro[2.5]octane-4-carboxylate (330 mg, 1.03 mmol, 1 eq) was dissolved in toluene, extra dry (5 mL) and malonic acid bis(2,4,6-trichlorophenyl) ester (526 mg, 1.14 mmol, 1.1 eq) was added at room temperature. The mixture was stirred for 2 hours at 80° C. The reaction mixture was evaporated and purified by flash chromatography (SiO₂, 25 g, MeOH in DCM 0 to 10%) to afford rac-tert-butyl 7-(2-hydroxy-4-oxo-pyrido[1,2-a]pyrimidin-7-yl)-4-azaspiro[2.5]octane-4-carboxylate (280 mg, 69.3%) as yellow solid. MS (ES+) m/z: 372.4 [(M+H)⁺].

Step 4: rac-tert-butyl 7-(6-amino-3-pyridyl)-4-azaspiro[2.5]octane-4-carboxylate (280 mg, 0.754 mmol, 1 eq) was dissolved in dichloromethane, extra dry (5 mL) and Et₃N (92 mg, 126 uL, 0.905 mmol, 1.2 eq) and p-toluenesulfonyl chloride (158 mg, 0.829 mmol, 1.1 eq) were added at room temperature. The mixture was stirred for 16 hours at room temperature. The reaction mixture was diluted with saturated NaHCO₃-solution and extracted two times with dichloromethane. The organic layers were washed with water and brine, dried over Na₂SO₄ and concentrated to dryness. The crude material was purified by flash chromatography on silica gel (25 g, AcOEt in Heptan 0 to 100%) to afford rac-tert-butyl 7-[4-oxo-2-(p-tolylsulfonyloxy)pyrido[1,2-a]pyrimidin-7-yl]-4-azaspiro[2.5]octane-4-carboxylate (325 mg, 80.4%) as white foam. MS (ES+) m/z: 526.4 [(M+H)⁺].

Step 5: In a 3-necked flask heated and dried under Argon, rac-tert-butyl 7-[4-oxo-2-(p-tolylsulfonyloxy)pyrido[1,2-a]pyrimidin-7-yl]-4-azaspiro[2.5]octane-4-carboxylate (120 mg, 0.228 mmol, 1 eq) and potassium carbonate (69 mg, 0.502 mmol, 2.2 eq) were charged and a degassed solution of 0.220 M (2,8-dimethylimidazo[1,2-b]pyridazin-6-yl)boronic acid (in solution in isopropyl acetate) (149 mg, 3.11 mL, 0.685 mmol, 3 eq) and degassed water (0.5 mL) were added. The mixture was purged 3 times with vacuum/argon cycles and then stirred at 55° C. under argon. Palladium(II) acetate (615 ug, 0.003 mmol, 0.012 eq) and tricyclohexylphosphine (1.5 mg, 0.005 mmol, 0.024 eq) were combined in separate flask flushed with argon and degassed isopropyl acetate (2 mL) was added. The solution was purged with argon again and this solution was then added via syringe to the first 3-necked flask at 55° C. After the addition, the reaction mixture was purged with vacuum/argon cycles and then stirred at 75° C. for 1 hour under argon. The reaction mixture was cooled down to 45° C. and 2 ml of water was added and it was stirred for 15 minutes at RT. The precipitate was filtered off and washed several times with water. The recovered solid was purified by flash chromatography (25 g SiO₂, MeOH in DCM, 0 to 10% for 20 min) to afford rac-tert-butyl 7-[2-(2,8-dimethylimidazo[1,2-b]pyridazin-6-yl)-4-oxo-pyrido[1,2-a]pyrimidin-7-yl]-4-azaspiro[2.5]octane-4-carboxylate (90 mg, 70.88%) as light yellow solid. MS (ES+) m/z: 501.5 [(M+H)⁺].

Step 6: rac-tert-butyl 7-[2-(2,8-dimethylimidazo[1,2-b]pyridazin-6-yl)-4-oxo-pyrido[1,2-a]pyrimidin-7-yl]-4-azaspiro[2.5]octane-4-carboxylate (80 mg, 0.152 mmol, 1 eq) was dissolved in extra dry dichloromethane (2 mL) and 4 M HCl in Dioxane (455 mg, 379 uL, 1.52 mmol, 10 eq) was added. The mixture was stirred for 2 hours at room temperature, then evaporated. The solid was diluted and basified with 1M Na₂CO₃-solution (pH>10) and extracted 3 times with DCM/MeOH 95/5. The organic layers were dried over Na₂SO₄ and concentrated to dryness. The crude material was purified by flash chromatography on silica gel (25 g, MeOH in DCM 5 to 15%) to afford rac-7-(4-azaspiro[2.5]octan-7-yl)-2-(2,8-dimethylimidazo[1,2-b]pyridazin-6-yl)pyrido[1,2-a]pyrimidin-4-one (45 mg, 72.5%) as light brown solid. MS (ES+) m/z: 401.3 [(M+H)⁺].

### Example 2

### rac-7-(4-azaspiro[2.5]octan-7-yl)-2-(8-methoxy-2-methyl-imidazo[1,2-b]pyridazin-6-yl)pyrido[1,2-a]pyrimidin-4-one

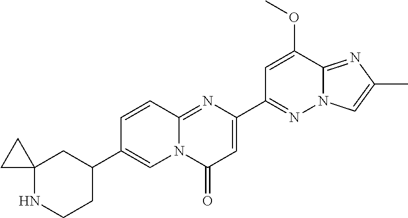 [SMILES: COc1cc(-c2cc(=O)n3cc(C4CCNC5(CC5)C4)ccc3n2)nn2cc(C)nc12]

Step 1: In a 3-necked flask, rac-tert-butyl 7-[4-oxo-2-(p-tolylsulfonyloxy)pyrido[1,2-a]pyrimidin-7-yl]-4-azaspiro[2.5]octane-4-carboxylate, described herein above in example 1 (80 mg, 0.152 mmol, 1 eq), potassium carbonate (84 mg, 0.609 mmol, 4 eq), [1,1′-bis(diphenylphosphino)ferrocene]dichloro-palladium(II) (11 mg, 0.015 mmol, 0.1 eq) and (8-methoxy-2-methyl-imidazo[1,2-b]pyridazin-6-yl)boronic acid (79 mg, 0.381 mmol, 2.5 eq) were dissolved in degassed acetonitrile (1.5 mL) and water (0.5 mL). After purging with argon, the mixture was stirred for 18 hours at 100° C. under argon. The reaction mixture was diluted with saturated NaHCO₃-solution and extracted two times with EtOAc. The organic layers were washed with water and brine, dried over Na₂SO₄ and concentrated to dryness. The crude material was purified by flash chromatography on silica gel (25 g, MeOH in DCM 0 to 5%) to afford rac-tert-butyl 7-[2-(8-methoxy-2-methyl-imidazo[1,2-b]pyridazin-6-yl)-4-oxo-pyrido[1,2-a]pyrimidin-7-yl]-4-azaspiro[2.5]octane-4-carboxylate (90 mg, 98.4%) as light brown waxy solid. MS (ES+) m/z: 517.5 [(M+H)⁺].

Step 2: rac-tert-butyl 7-[2-(8-methoxy-2-methyl-imidazo[1,2-b]pyridazin-6-yl)-4-oxo-pyrido[1,2-a]pyrimidin-7-yl]-4-azaspiro[2.5]octane-4-carboxylate (90 mg, 0.150 mmol, 1 eq) was dissolved in dichloromethane, extra dry (1 mL) and TFA (427 mg, 288 uL, 3.75 mmol, 25 eq) was added. The mixture was stirred for 2 hours at room temperature. The reaction mixture was concentrated, then rediluted with 2 ml of toluene and evaporated again to dryness. The brown crude material was diluted in DCM/MeOH 95/5 (20 mL) and water (15 mL) and neutralised dropwise with 25% Ammonia in water (1 mL). The aqueous phase (pH=12) was extracted 3 times with DCM/MeOH 95/5. The organic layers were combined, dried over Na₂SO₄ and concentrated to dryness. The crude material was purified by flash chromatography on silica gel (12 g, MeOH in DCM 0 to 15%) to afford rac-7-(4-azaspiro[2.5]octan-7-yl)-2-(8-methoxy-2-methyl-imidazo[1,2-b]pyridazin-6-yl)pyrido[1,2-a]pyrimidin-4-one (45 mg, 71.4%) as off-white solid. MS (ES+) m/z: 417.3 [(M+H)⁺].

### Example 3

### rac-7-(4-azaspiro[2.5]octan-7-yl)-2-[2-methyl-8-(trifluoromethyl)imidazo[1,2-b]pyridazin-6-yl]pyrido[1,2-a]pyrimidin-4-one

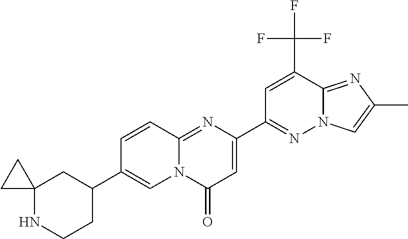 [SMILES: Cc1cn2nc(-c3cc(=O)n4cc(C5CCNC6(CC6)C5)ccc4n3)cc(C(F)(F)F)c2n1]

Step 1: In a 3-necked flask, rac-tert-butyl 7-[4-oxo-2-(p-tolylsulfonyloxy)pyrido[1,2-a]pyrimidin-7-yl]-4-azaspiro[2.5]octane-4-carboxylate, described herein above in example 1 (80 mg, 0.152 mmol, 1 eq), potassium carbonate (46 mg, 0.335 mmol, 2.2 eq) and [2-methyl-8-(trifluoromethyl)imidazo[1,2-b]pyridazin-6-yl]boronic acid (93 mg, 0.381 mmol, 2.5 eq) were charged and dissolved with degassed isopropyl acetate (2 mL) and water (0.5 mL). It was purged 3 times with vacuum/argon cycles and then stirred at 55° C. under argon. Palladium(II) acetate (410 ug, 0.002 mmol, 0.012 eq) and tricyclohexylphosphine (1.0 mg, 0.004 mmol, 0.024 eq) were combined in another flask flushed with argon and degassed isopropyl acetate (2 mL) was added. The solution was purged with argon again and this solution was then added via syringe to the 1st 3-necked flask at 55° C. After the addition, the reaction mixture was purged with vacuum/argon cycles and then stirred at 75° C. for 1 hour under argon. The reaction mixture was cooled down and quenched with 2 ml of water. The mixture was diluted with saturated NaHCO₃ solution and AcOEt. The organic phase was separated, washed with brine, dried over Na₂SO₄ and evaporated. The crude material was purified by flash chromatography (25 g SiO₂, MeOH in DCM, 0 to 5%) to afford rac-tert-butyl 7-[2-[2-methyl-8-(trifluoromethyl)imidazo[1,2-b]pyridazin-6-yl]-4-oxo-pyrido[1,2-a]pyrimidin-7-yl]-4-azaspiro[2.5]octane-4-carboxylate (80 mg, 90.%) as yellow solid. MS (ES+) m/z: 555.4 [(M+H)⁺].

Step 2: In analogy to example 2 step 2, from rac-tert-butyl 7-[2-[2-methyl-8-(trifluoromethyl)imidazo[1,2-b]pyridazin-6-yl]-4-oxo-pyrido[1,2-a]pyrimidin-7-yl]-4-azaspiro[2.5]octane-4-carboxylate was obtained rac-7-(4-azaspiro[2.5]octan-7-yl)-2-[2-methyl-8-(trifluoromethyl)imidazo[1,2-b]pyridazin-6-yl]pyrido[1,2-a]pyrimidin-4-one (36 mg, 61%) as a yellow solid. MS (ES+) m/z: 455.3 [(M+H)⁺].

### Example 4

### rac-7-(4-azaspiro[2.5]octan-7-yl)-2-(2,7-dimethyloxazolo[5,4-b]pyridin-5-yl)pyrido[1,2-a]pyrimidin-4-one

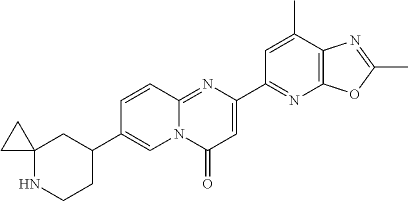 [SMILES: Cc1nc2c(C)cc(-c3cc(=O)n4cc(C5CCNC6(CC6)C5)ccc4n3)nc2o1]

Step 1: In analogy to example 2 step 1, from rac-tert-butyl 7-[4-oxo-2-(p-tolylsulfonyloxy)pyrido[1,2-a]pyrimidin-7-yl]-4-azaspiro[2.5]octane-4-carboxylate, described herein above in example 1 and 2,7-dimethyl-5-(4,4,5,5-tetramethyl-1,3,2-dioxaborolan-2-yl)oxazolo[5,4-b]pyridine was obtained rac-tert-butyl 7-[2-(2,7-dimethyloxazolo[5,4-b]pyridin-5-yl)-4-oxo-pyrido[1,2-a]pyrimidin-7-yl]-4-azaspiro[2.5]octane-4-carboxylate (24 mg, 35%) as an off-white solid. MS (ES+) m/z: 502.4 [(M+H)⁺].

Step 2: In analogy to example 2 step 2, from rac-tert-butyl 7-[2-(2,7-dimethyloxazolo[5,4-b]pyridin-5-yl)-4-oxo-pyrido[1,2-a]pyrimidin-7-yl]-4-azaspiro[2.5]octane-4-carboxylate was obtained rac-7-(4-azaspiro[2.5]octan-7-yl)-2-(2,7-dimethyloxazolo[5,4-b]pyridin-5-yl)pyrido[1,2-a]pyrimidin-4-one (15 mg, 85%) as an off-white solid. MS (ES+) m/z: 402.3 [(M+H)⁺].

### Example 5

### rac-7-(4-azaspiro[2.5]octan-7-yl)-2-(2-chloroimidazo[1,2-b]pyridazin-6-yl)pyrido[1,2-a]pyrimidin-4-one

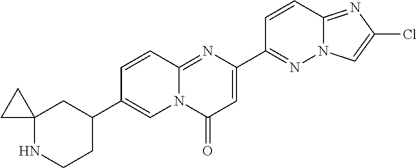 [SMILES: O=c1cc(-c2ccc3nc(Cl)cn3n2)nc2ccc(C3CCNC4(CC4)C3)cn12]

Step 1: In analogy to example 2 step 1, from rac-tert-butyl 7-[4-oxo-2-(p-tolylsulfonyloxy)pyrido[1,2-a]pyrimidin-7-yl]-4-azaspiro[2.5]octane-4-carboxylate, described herein above in example 1 and 2-chloro-6-(4,4,5,5-tetramethyl-1,3,2-dioxaborolan-2-yl)imidazo[1,2-b]pyridazine was obtained rac-tert-butyl 7-[2-(2-chloroimidazo[1,2-b]pyridazin-6-yl)-4-oxo-pyrido[1,2-a]pyrimidin-7-yl]-4-azaspiro[2.5]octane-4-carboxylate (15 mg, 17%) as a light brown solid. MS (ES+) m/z: 507.3 [(M+H)⁺].

Step 2: In analogy to example 2 step 2, from rac-tert-butyl 7-[2-(2-chloroimidazo[1,2-b]pyridazin-6-yl)-4-oxo-pyrido[1,2-a]pyrimidin-7-yl]-4-azaspiro[2.5]octane-4-carboxylate was obtained rac-7-(4-azaspiro[2.5]octan-7-yl)-2-(2-chloroimidazo[1,2-b]pyridazin-6-yl)pyrido[1,2-a]pyrimidin-4-one (9 mg, 81%) as a yellow solid. MS (ES+) m/z: 407.3 [(M+H)⁺].

### Example 6

### Homogeneous Time Resolved Fluorescence for HTT Lowering

The HTRF assay was adapted from Weiss et al. (Analytical Biochemistry Volume 395, Issue 1, 1 Dec. 2009, Pages 8-15 and Analytical Biochemistry Volume 410, 2011, Pages 304-306) to cells from GENEAe020-A cell line (https.//hpscreg.eu/cell-line/GENEAe020-A).

Compounds were tested for the effect of mutant HTT levels in Huntington patient human cells (GENEAe020-A cell line) using Homogeneous Time Resolved Fluorescence (HTRF) directed towards mutant HTT protein (mHTT). The GENEAe020-A cell line was derived by Genea Biocells from human blastocysts of HD donors. After assessing viability, cells were plated into 384 well collagen coated plates in growth media. Once cells adhered, media was removed and test compounds dissolved in DMSO were diluted with buffer solution and added to the adherent cells. Controls included experiments with no cells, DMSO with no compound, and Hsp90 inhibitor control. Cells were incubated with compounds and controls for 48 hours. Then, the cells were lysed and transferred to an assay plate containing HTRF labeled monoclonal antibodies developed by Paul Patterson (Ko et al., Brain Research Bulletin, Volume 56, Numbers 3 and 4, 2001, Pages 319-329) which recognize specific areas of the HTT protein. The terbium labeled “donor” antibody (2B7) binds to the N-terminus of the HTT protein and the Alexa488 labeled “acceptor” antibody (MW1) is specific for the polyglutamine region of the protein. Binding of the acceptor labeled antibody is more efficient for the extended polyglutamine repeats of mutant HTT protein which translates into a signal boost which enables the specific measurement of mutant HTT protein level. The HTRF donor and acceptor detection reagents were incubated with the cell lysate and the ratio between the signals of the two fluorophores is indicative of the relative quantities of mHTT.

The results of this assay are provided in Table 8 below. Table 8 provides the EC₅₀ (half maximal effective concentration) values for the reduction of mHTT obtained for particular examples of the present invention as measured by HTRF assay (data shown below is mean from three replicates).

| Exam. | HTRF mHTT EC₅₀ (μM) |
|---|---|
| 1 | 0.031 |
| 2 | 0.021 |
| 3 | 0.04 |
| 4 | 0.049 |
| 5 | 0.259 |

1. A compound of formula (I) 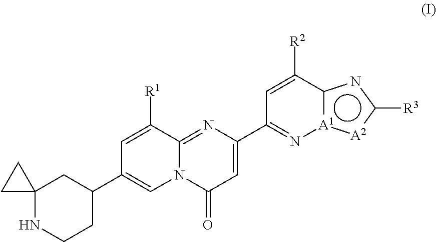
wherein
R¹ is hydrogen or C₁₋₈ alkyl;
R² is hydrogen, halogen, C₁₋₈ alkyl, C₁₋₈ alkoxy or C₁₋₈ haloalkyl;
R³ is hydrogen, C₁₋₈ alkyl or halogen;
A¹ is —N— or —C—; and
A² is —CH— or —O—; with the proviso that if A¹ is —N—, then A² is —CH—; and the proviso that if A¹ is —C—, then A² is —O—;
or a pharmaceutically acceptable salt thereof.

2. A compound according to claim 1, wherein R¹ is hydrogen or methyl.

3. A compound according to claim 1, wherein R¹ is hydrogen.

4. A compound according to claim 1, wherein R² is hydrogen, C₁₋₈ alkyl, C₁₋₈ alkoxy or C₁₋₈ haloalkyl.

5. A compound according to claim 1, wherein R² is hydrogen, methyl, methoxy or trifluoromethyl.

6. A compound according to claim 1, wherein R³ is C₁₋₈ alkyl or halogen.

7. A compound according to claim 1, wherein R³ is methyl or chloro.

8. A compound according to claim 1, wherein A₁ is —N—, and A² is —CH—.

9. A compound according to claim 1, wherein A₁ is —C—, and A₂ is —O—.

10. A compound of formula (I) according to claim 1 selected from 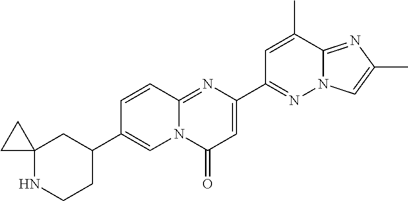 [SMILES: Cc1cn2nc(-c3cc(=O)n4cc(C5CCNC6(CC6)C5)ccc4n3)cc(C)c2n1]
rac-7-(4-azaspiro[2.5]octan-7-yl)-2-(2,8-dimethylimidazo[1,2-b]pyridazin-6-yl)pyrido[1,2-a]pyrimidin-4-one; 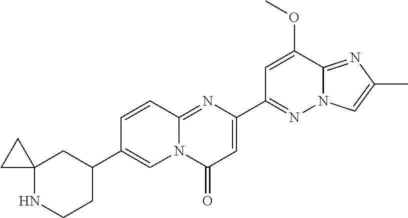 [SMILES: COc1cc(-c2cc(=O)n3cc(C4CCNC5(CC5)C4)ccc3n2)nn2cc(C)nc12]
rac-7-(4-azaspiro[2.5]octan-7-yl)-2-(8-methoxy-2-methyl-imidazo[1,2-b]pyridazin-6-yl)pyrido[1,2-a]pyrimidin-4-one; 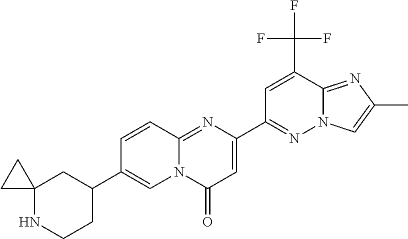 [SMILES: Cc1cn2nc(-c3cc(=O)n4cc(C5CCNC6(CC6)C5)ccc4n3)cc(C(F)(F)F)c2n1]
rac-7-(4-azaspiro[2.5]octan-7-yl)-2-[2-methyl-8-(trifluoromethyl)imidazo[1,2-b]pyridazin-6-yl]pyrido[1,2-a]pyrimidin-4-one; 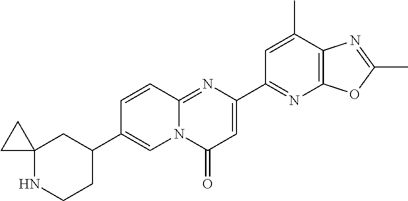 [SMILES: Cc1nc2c(C)cc(-c3cc(=O)n4cc(C5CCNC6(CC6)C5)ccc4n3)nc2o1]
rac-7-(4-azaspiro[2.5]octan-7-yl)-2-(2,7-dimethyloxazolo[5,4-b]pyridin-5-yl)pyrido[1,2-a]pyrimidin-4-one; and 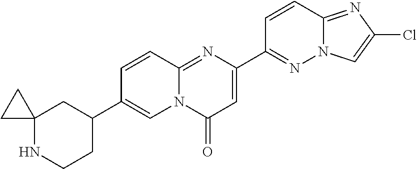 [SMILES: O=c1cc(-c2ccc3nc(Cl)cn3n2)nc2ccc(C3CCNC4(CC4)C3)cn12]
rac-7-(4-azaspiro[2.5]octan-7-yl)-2-(2-chloroimidazo[1,2-b]pyridazin-6-yl)pyrido[1,2-a]pyrimidin-4-one;
or a racemic mixture or its corresponding enantiomers thereof,
or a pharmaceutically acceptable salt thereof.

11. A process for the preparation of a compound according to claim 1, comprising at least one of the following steps:
(a) reacting a compound of formula (B1) 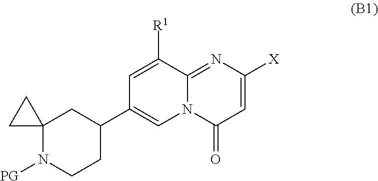
with a compound of formula (B2) 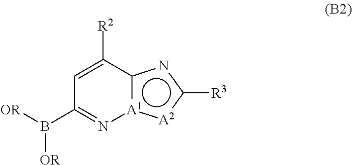
in a suitable solvent in the presence of a base and a suitable palladium catalyst, wherein n is 0 or 1, X is O-tosylate, O-triflate, O-mesylate or halogen, and wherein in —B(OR)₂ each R is independently selected from hydrogen and C₁₋₈ alkyl, or —B(OR)₂ is optionally substituted dioxaborolanyl, to arrive at a compound of formula (B3) 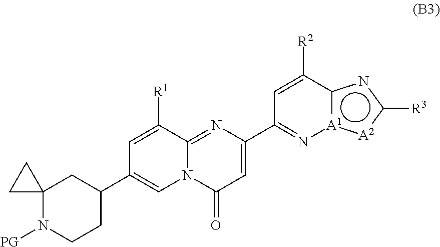
(b) reacting the compound of formula (B3), in a suitable solvent and in presence of an acid to yield the compound of formula (I) 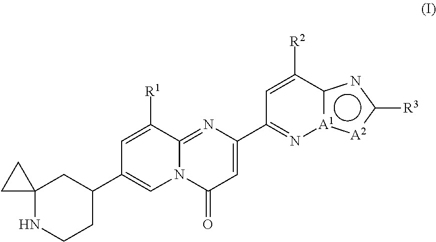
wherein in the process R¹, R², R³, A₁ and A₂ are as defined in claim 1, and PG is a protecting group.

12. A compound according to claim 1, when manufactured according to a process of claim 11.

13. (canceled)

14. A pharmaceutical composition comprising a compound according to claim 1 and a therapeutically inert carrier.

15. (canceled)

16. (canceled)

17. (canceled)

18. A method of treating Huntington's disease, the method comprising administering an effective amount of a compound according to claim 1 to a patient in need thereof.

19. (canceled)
